SEQUENCE LISTING (1 sequence)

SEQ ID NO: 1 - DNA, 43 bp, Mycobacterium tuberculosis
  misc_feature (11)..(11): n is a, c, g, or t
  misc_feature (14)..(14): n is a, c, g, or t
  misc_feature (16)..(16): n is a, c, g, or t
  misc_feature (18)..(26): n is a, c, g, or t
  misc_feature (28)..(28): n is a, c, g, or t
  misc_feature (30)..(30): n is a, c, g, or t
  misc_feature (33)..(33): n is a, c, g, or t
cagacgacagnaancncnnnnnnnnngngnttntcgctgtctg

(19) United States
(12) Patent Application Publication
(10) Pub. No.: US 2022/0380786 A1
(43) Pub. Date: Dec. 1, 2022

(54) SYSTEM AND METHOD FOR COMBATING MYCOBACTERIUM TUBERCULOSIS INFECTIONS

(71) Applicant: Tata Consultancy Services Limited, Mumbai (IN)

(72) Inventors: SHARMILA SHEKHAR MANDE, Pune (IN); SWADHA ANAND, Pune (IN); PREETHI ALAGARAI SAMPATH, Pune (IN)

(73) Assignee: Tata Consultancy Services Limited, Mumbai (IN)

(21) Appl. No.: 17/596,186

(22) PCT Filed: Jun. 4, 2020
(86) PCT No.: PCT/IB2020/055274

§ 371 Date: Dec. 3, 2021

(30) Foreign Application Priority Data
Jun. 6, 2019 (IN) 201921022522

(51) Int. Cl. C12N 15/74; C12N 9/22; A61K 38/46; A61K 38/16; C12N 15/10; C12Q 1/689; C12N 15/11

(52) U.S. Cl. CPC C12N 15/74; C12N 9/22; A61K 38/465; A61K 38/164; C12N 15/1072; C12Q 1/689; C12N 15/111; C12N 2310/20

(57) ABSTRACT

Mycobacterium tuberculosis is the most common pathogenic agent responsible for tuberculosis (TB) infection. Over a period of time, the methods used for combating TB have become more challenging by the prevalence of multi-drug resistant and extensively drug resistant strains. The disclosure relates generally to method and system for combating infections due to Mycobacterium tuberculosis. The system provides strategies to combat pathogenic infections caused by multi-drug resistant (MDR) and extensively drug resistant (XDR) strains of Mycobacterium tuberculosis. The strategy involves identifying potential target sites in a pathogen, which can be utilized to compromise its multiple virulence or essential functions at the same time. The present disclosure utilizes the fact that a conserved stretch of nucleotide repeat sequence occurring multiple times on a pathogen genome in genomic neighborhood of genes encoding virulence factors for pathogen survival can be targeted to disrupt the overall genetic machinery of the pathogen.

### CROSS-REFERENCE TO RELATED APPLICATIONS AND PRIORITY

The present application claims priority from Indian provisional application no. 201921022522, filed on Jun. 6, 2019. The entire contents of the aforementioned application are incorporated herein by reference.

### TECHNICAL FIELD

The embodiments herein generally relates to the field of Mycobacterium tuberculosis infections, and, more particularly, to a method and system for combating the problem of multidrug resistance resulting due to infection of Mycobacterium tuberculosis.

### BACKGROUND

Tuberculosis (TB) is among the most common infectious disease as well as one of the deadliest diseases in the world. According to CDC (Center of Disease Control and Prevention) one fourth of the world population is affected by TB. It is also the prime cause of death in patients affected by HIV. Mycobacterium tuberculosis is the most common pathogenic agent responsible for TB infection. Mycobacterium tuberculosis infection is spread through air and often infects the lung in the patients.

The treatment methods are made more challenging by the prevalence of multi-drug resistant (MDR-TB) and extensively drug resistant (XDR-TB) strains. The most commonly used antibiotics in tuberculosis induced by Mycobacterium tuberculosis are Isoniazid, Rifampin (Rifadin, Rimactane), Ethambutol (Myambutol), Pyrazinamide etc. Most MDR-TB strains are resistant to both first line drugs Isoniazid and Rifampin and XDR-TB strains are resistant to isoniazid and rifampin, plus any fluoroquinolone and at least one of three injectable second-line drugs such as amikacin, kanamycin, or capreomycin etc. This makes the available treatment options for such drug-resistant strains much less effective. Therefore, CDC classifies drug-resistant Mycobacterium tuberculosis as serious level threat.

Additional problems arise which pertain to formation of biofilms in Mycobacterium which allows them to evade antibiotics. Several studies have shown that biofilm formation inhibitors (like several enzymes which degrade the matrix) as well as quorum quenchers (prevent biofilm formation) can prove useful in this regard. Despite utilizing these inhibitors Mycobacterium still escapes the antibiotics and lead to relapse once the treatment is stopped.

Several side effects and cross-reactivity is observed in present drugs used in the treatment of Mycobacterium tuberculosis. Most of the antibiotics land up killing the beneficial human microbiome also.

Further in one of the prior art is using repetitive DNA sequence specific for Mycobacterium tuberculosis for the diagnosis of tuberculosis. The repeat sequences specific to Mycobacterium species have been identified on the pathogen genome, however these sequences may not be ideal candidates for therapeutic purposes. Also, they have been used only in diagnostic aspects of Mycobacterial infections.

One type of repeat sequences identified on the Mycobacterium tuberculosis genome constitutes mobile elements capable of transferring gene elements between bacterial species. Although dispersed across the genome, these repeat sequences cannot be used as targets because they might be transferred to other bacteria. Further, the chances of mutations in these sequences might also be higher. In addition to that, another type of repeat sequence identified in Mycobacterium tuberculosis and Mycobacterium leprae are tandem repeat sequences that are clustered together in one part of the Mycobacterium genome and are not dispersed throughout the genome. Hence, targeting such sequences only cleave a part of the pathogen genome which can be repaired by DNA repair machinery available in the bacterial genome.

### SUMMARY

Embodiments of the present disclosure present technological improvements as solutions to one or more of the above-mentioned technical problems recognized by the inventors in conventional systems. For example, in one embodiment the system is provided for combating infections due to Mycobacterium tuberculosis. The system comprises a sample collection module, a pathogen detection and DNA extraction module, a sequencer, one or more hardware processors, an administration module and an efficacy module. The sample collection module obtains a sample from an infected area. The pathogen detection and DNA extraction module isolates DNA from the obtained sample using one of a laboratory methods. The sequencer sequences the isolated DNA. The memory in communication with the one or more hardware processors, wherein the one or more first hardware processors are configured to execute programmed instructions stored in the one or more first memories, to: identify a set of nucleotide repeat sequences in the sequenced DNA which are occurring more than a predefined number of times in the Mycobacterium Tuberculosis; identify a set of neighborhood genes present upstream and downstream of the set of nucleotide repeat sequences; annotate the set of neighborhood genes according to their functional roles in their respective pathogen based on their involvement in pathways in the identified set of neighborhood genes; test the presence of a secondary structure in the identified set of nucleotide repeat sequences. The administration module prepares and administers an engineered polynucleotide construct on the infected area to combat the infections due to the Mycobacterium tuberculosis, wherein the engineered polynucleotide construct is comprising: one or more of a set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001, a first enzyme capable of nicking and cleaving the identified set of nucleotide repeat sequences, and a second enzyme capable of removal of a set of neighborhood genes flanking the set of nucleotide repeat sequences. The efficacy module checks the efficacy of the administered engineered polynucleotide construct to combat the Mycobacterium tuberculosis after a predefined time period; and re-administers the engineered polynucleotide construct if the Mycobacterium tuberculosis are still present in the infected area post administering.

In another embodiment, a method for combating infections due to Mycobacterium Tuberculosis is provided. Initially, a sample is obtained from an infected area. Further, DNA/RNA is isolated and extracted from the obtained sample using one of a laboratory method. Later, the isolated DNA/RNA is sequenced using a sequencer. In the next step, a set of nucleotide repeat sequences is identified in the sequenced DNA which are occurring more than a predefined number of times in the Mycobacterium Tuberculosis. Further, a set of neighborhood genes present upstream and downstream of the set of nucleotide repeat sequences is identified. Later, the set of neighborhood genes is annotated according to their functional roles in their respective pathogen based on their involvement in pathways in the identified set of neighborhood genes. In the next step, the presence of a secondary structure is tested in the identified set of nucleotide repeat sequences. Later, an engineered polynucleotide construct is prepared and administered on the infected area to combat the infections due to the Mycobacterium tuberculosis, wherein the engineered polynucleotide construct is comprising: one or more of a set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001, a first enzyme capable of nicking and cleaving the identified set of nucleotide repeat sequences, and a second enzyme capable of removal of a set of neighborhood genes flanking the set of nucleotide repeat sequences. In the next step, the efficacy of the administered engineered polynucleotide construct is checked to combat the Mycobacterium tuberculosis after a predefined time period. And finally, the engineered polynucleotide construct is re-administered if the Mycobacterium tuberculosis are still present in the infected area post administering.

The target sites or nucleotide repeat sequences in this disclosure refer to nucleotide sequences which repeat a minimum number of ten times within the genome of the candidate pathogen/pathogens which are identified in an infected site from which the sample is collected. These nucleotide repeat sequences can be targeted in order to debilitate the pathogen. The mentioned nucleotide repeat sequence/sequences is selected if it occurs more than 10 times in all the strains of the candidate specie or genus to which the candidate pathogen/pathogens identified in an infected site belong. The nucleotide repeat sequence is selected such that it does not occur more than twice in genomes of strains belonging to any other genus than that of the candidate pathogen and does not occur more than twice within the genome of the host.

In yet another aspect, one or more non-transitory machine readable information storage mediums comprising one or more instructions which when executed by one or more hardware processors cause combating infections due to Mycobacterium Tuberculosis is provided. Initially, a sample is obtained from an infected area. Further, DNA/RNA is isolated and extracted from the obtained sample using one of a laboratory method. Later, the isolated DNA/RNA is sequenced using a sequencer. In the next step, a set of nucleotide repeat sequences is identified in the sequenced DNA which are occurring more than a predefined number of times in the Mycobacterium Tuberculosis. Further, a set of neighborhood genes present upstream and downstream of the set of nucleotide repeat sequences is identified. Later, the set of neighborhood genes is annotated according to their functional roles in their respective pathogen based on their involvement in pathways in the identified set of neighborhood genes. In the next step, the presence of a secondary structure is tested in the identified set of nucleotide repeat sequences. Later, an engineered polynucleotide construct is prepared and administered on the infected area to combat the infections due to the Mycobacterium tuberculosis, wherein the engineered polynucleotide construct is comprising: one or more of a set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001, a first enzyme capable of nicking and cleaving the identified set of nucleotide repeat sequences, and a second enzyme capable of removal of a set of neighborhood genes flanking the set of nucleotide repeat sequences. In the next step, the efficacy of the administered engineered polynucleotide construct is checked to combat the Mycobacterium tuberculosis after a predefined time period. And finally, the engineered polynucleotide construct is re-administered if the Mycobacterium tuberculosis are still present in the infected area post administering.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates a block diagram of a system for combating infections due to Mycobacterium tuberculosis according to an embodiment of the present disclosure.

FIG. 2A shows nucleotide repeat sequences along with neighborhood genes in the Mycobacterium tuberculosis genome according to an embodiment of the disclosure.

FIG. 3 shows components of a construct containing multiple target nucleotide sequences capable of combating Mycobacterium tuberculosis infections according to an embodiment of the disclosure.

FIG. 4 shows targeting of nucleotide repeat sequences in pathogen genomes according to an embodiment of the disclosure.

FIG. 5 shows enzymatic cleavage in the Mycobacterium tuberculosis genome according to an embodiment of the disclosure.

FIG. 6A-6B is a flowchart illustrating the steps involved in combating infections due to Mycobacterium tuberculosis according to an embodiment of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

### Glossary— Terms Used in the Embodiments

The expression “nucleotide repeat sequences” or “repeated nucleotide sequences” or “the set of nucleotide repeats” or “repeated sequence regions” or “repeat element” or “target sequences” or “target sites” or “similar sequence stretches” or “target nucleotide repeat sequence” or “conserved stretch of nucleotide sequences” in the context of the present disclosure refers to nucleotide sequences which have been repeated multiple times in a sequence of DNA extracted from a sample obtained from the infected area or within nucleotide sequence obtained for a genomic sequence of a pathogen or genomic sequences of strains belonging to a pathogenic genus or specie.

The term “metagenome” refers to the genetic material derived directly from the infected site and can be considered representative of overall microorganisms present in a sample collected from an environment. The information about metagenome and its taxonomic constitution is obtained by either sequencing the genes considered as markers for different taxa (For example 16S rRNA), amplifying genes of interest using specific primers through methods like but not limited to Polymerase Chain Reaction (PCR). This information can also be obtained by whole genome sequencing of the obtained environmental or metagenomic sample. The sample collected from the environment is referred to from now on as metagenomic sample.

The term “identified nucleotide repeat sequence is dispersed across distant locations in the pathogen genome” refers to the fact that the nucleotide sequences identified in this method are spread at distant locations across the pathogen genome and is not clustered together at one particular location alone on the genome.

In this disclosure, the terms “distant location” or “distinct location” or “dispersed location” refer to locations of two nucleotide repeat sequences that are separated by >10000 base pairs. Nucleotide repeat regions having distance less than 10000 base pairs between their locations have been considered as clustered repeats.

The expression “candidate genus” or ‘candidate pathogen’ refers to the genus, specie or pathogen in which the nucleotide repeat sequence is identified and is used as a target sequence/site.

The term “commensal” refers to microbe/microbes which are considered beneficial to the host or cause no harm to the host.

The term ‘pathogen’ refers to microbe/microbes which cause a disease in host.

The term ‘host’ refers to either a living organism or an environmental site. In an embodiment, ‘host’ may refer to human, animal or plant in which a pathogenic infection may be observed.

The term ‘non-culturable’ refers to microbes that cannot be grown in a laboratory settings because the ideal conditions and media for their growth is not well characterized. Such microbes can be analyzed by culture independent methods discussed in various embodiments of the disclosure.

Majority of the existing methods for combating pathogens focus on silencing specific genes in order to curtail their expression. Targeting single functional aspects of bacteria often is not sufficient as bacteria might mutate the targets and develop resistance to the therapeutic intervention. To overcome the drawbacks of the existing methods, the present system and method deals with identifying and targeting multiple copies of a nucleotide repeat sequence at distant locations on the genome as well as the important functional genes flanking this sequence. Therefore, the method allows to debilitate multiple important functions of the pathogen simultaneously. The important functional genes in this disclosure refer to the genes in pathogens which encode for proteins which are critical for survival, pathogenicity, interaction with the host, adherence to the host or for the virulence of bacteria. Development of resistance in pathogens to the method mentioned in this disclosure is difficult as the pathogen will have to bring about multiple mutations in distant locations. The present disclosure includes targeting multiple virulence and essential proteins of pathogens. The method may also include targeting various other proteins performing important functions (metabolism, host interactions, pathogenicity etc.) in bacteria.

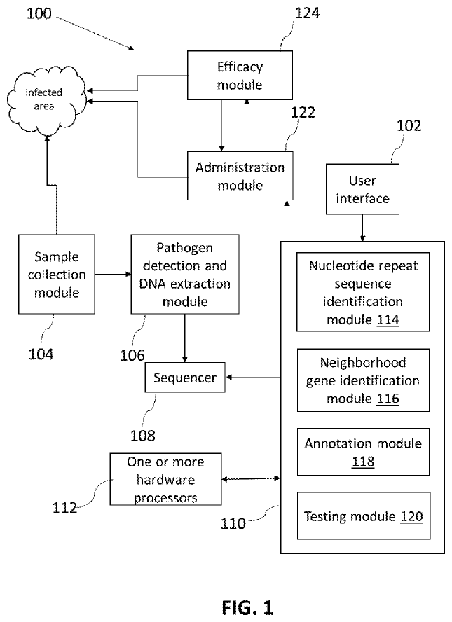

Referring now to the drawings, and more particularly to FIG. 1 through FIG. 6B, where similar reference characters denote corresponding features consistently throughout the figures, there are shown preferred embodiments and these embodiments are described in the context of the following exemplary system and/or method.

According to an embodiment of the disclosure, a system 100 for combating infections due to Mycobacterium tuberculosis is shown in the block diagram of FIG. 1. The system 100 is configured to provide strategies to combat pathogenic infections caused by multi-drug resistant (MDR) and extensively drug resistant (XDR) strains of Mycobacterium tuberculosis. The strategy involves identifying potential target sites in a pathogen, which can be utilized to compromise its multiple virulence or essential functions at the same time. The present disclosure utilizes the fact that a conserved stretch of nucleotide repeat sequence occurring multiple times on a pathogen genome in genomic neighbourhood of genes encoding virulence factors or in vicinity of genes essential for pathogen survival encoded within the genome of the candidate pathogen can be targeted to disrupt the overall genetic machinery of the pathogen. These nucleotide repeat sequences might also lie in the neighborhood of genes which perform other critical functions in a pathogen.

In the present disclosure genomic neighbourhood or vicinity or ‘flanking genes’ refers to regions lying within a predefined number of genes to the selected nucleotide repeat sequence (or its reverse complement) on the nucleotide sequence of the candidate pathogen genome or within a distance of predefined number of bases with respect to the selected nucleotide repeat sequence (or its reverse complement) on the nucleotide sequence of the pathogen genome. The flanking genes are found on each strand on pathogen genomic DNA. In an embodiment the genomic neighbourhood or flanking genes may comprise of 10 genes lying on either side of nucleotide repeat sequence or its reverse complement in terms of its location on the pathogen genome. The reverse complement of target sequence is obtained by interchanging letters A and T and interchanging letters C and G between target and complement sequence.

A conserved stretch of sequence refers to a nucleotide repeat sequence which occurs within all pathogenic genomes belonging to a candidate genus. Another important factor would be occurrence of these sequences only in genomic sequences of pathogenic strains of the candidate pathogen and minimum cross reactivity with the commensals (belonging to same candidate genus or other genera) as well as the host. Cross reactivity, in this disclosure, refers to the occurrence of these conserved stretches of nucleotide repeat sequences more than twice in genera/specie other than the candidate genus or more than twice within commensal bacteria belonging to the candidate genus/specie for which this sequence is being utilized as a target. The nucleotide repeat sequence should not occur more than twice in the host genome also. Further, the identified potential target sites in pathogen are not specific to a single strain of the pathogen. In most cases, metagenomic samples contain bacteria whose strain level information cannot be obtained. Thus, the method can be utilized to target all pathogens in the given species of the bacteria and is not hindered by the absence of strain level information. The method disclosed in the present disclosure targets repeats sequences that are dispersed across distinct genomic locations in the pathogen allowing cleavage of the genomes of pathogenic strains of Mycobacteria in multiple places simultaneously. The method can be used as primarily in disinfection and therapeutic purposes.

According to an embodiment of the disclosure, the system 100 consists of a user interface 102, a sample collection module 104, a pathogen detection and DNA extraction module 106, a sequencer 108, a memory 110 and a processor 112 as shown in FIG. 1. The processor 112 is in communication with the memory 110. The memory 110 further includes a plurality of modules for performing various functions. The memory 110 may include a nucleotide repeat sequence identification module 114, a neighborhood gene identification module 116, an annotation module 118 and a testing module 120. The system 100 further comprises an administration module 122 and an efficacy module 124 as shown in the block diagram of FIG. 1.

According to an embodiment of the disclosure, the sample is collected from the infected area using the sample collection module 104. In this module, the method utilized for extracting samples from the infected sites depends largely on the site of infection. In one embodiment, where the infection of the lung is caused by Mycobacterium tuberculosis, sample collection from the fluids in the lung due to the infection could be done by one of the following methods such as bronchoalveolar lavage collection, bronchial brushings, endobronchial biopsies and nasal scrape etc. In another embodiment, in case of infection in the upper respiratory tract sample collection from lung can be performed by oropharyngeal (OP) and nasopharyngeal (NP) swabs and sputum collection.

In an embodiment where the site of infection can also be an environment such as soil, air, water or surfaces. Sample collection from a surface can be performed using a sterile swab. Dry swabs may be recommended for wet surfaces and wet swabs are recommended for dry surfaces. Swabbing of the test surface may be performed by rolling the swab lightly back and forth. Water and soil samples may be collected from the environmental site of infection and sent for further procedure. Air samples can also be collected to identify the presence of air borne pathogen. Volumetric air samples for culture analyses can be taken by impacting a known volume of air onto a suitable growth medium. Any other laboratory accepted method of sample extraction and/or collection from environment as well as living organisms is within the scope of this invention. In another example, the samples obtained from infected area is one or more of fecal matter, blood, urine, tissue biopsy, hospital surfaces or environmental samples.

DNA/RNA is isolated and then extracted from the sample using laboratory standardized protocol using the pathogen detection and DNA extraction module 106 and sequencing is performed using the sequencer 108. It should be appreciated, that the bacterial cells are isolated from the extracted sample before being presented to pathogen detection and DNA extraction module 106 in cases where the pathogen is known to be culturable. In case of non-culturable pathogen, the collected samples are directly processed to the pathogen detection and DNA extraction module 106, DNA/RNA is isolated and extracted from the sample using laboratory standardized protocols using the pathogen detection and DNA extraction module 106 and sequencing is performed using the sequencer 108. The nucleotide sequences obtained after sequencing of extracted DNA/RNA sequences are then provided to the processor 112 using the user interface 102. The nucleotide sequences can be obtained for 16S rRNA, a nucleotide sequence encoding for any particular gene of interest being amplified, or sequences corresponding to DNA fragments corresponding to whole genome sequencing or shotgun sequencing. In one embodiment, DNA/RNA can be extracted using DNA isolation and extraction kits such as miniprep and other methods standardized in laboratory setups. The extracted DNA is then provided into the sequencer 108 and the sequences so obtained are fed into the processor 112 using the user interface 102. The user interface 102 is operated by a user. The user interface 102 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite.

The pathogen detection and DNA extraction module 106 is also configured to utilize experimental techniques to detect pathogens present in an infected site. The use of any laboratory acceptable methods of detecting presence of pathogens present at the infected site is within scope of the disclosure. In one embodiment, presence of viable living cells can be detected by utilizing presence of bacterial mRNA which has a short half-life and will not exist once the cells are dead. This mRNA based method may involve identifying antigen/protein specific for the pathogen which can be utilized as a marker for that pathogen and produced by the pathogen in abundance and the corresponding gene on the pathogen genome can be obtained (For example alpha antigen in Mycobacterium tuberculosis). The mRNA corresponding to expression of these genes can be detected using techniques like but not limited to polymerase chain reaction (RT-PCR) assays or reverse transcriptase strand displacement amplification (RT-SDA) assays. In another embodiment, expression of proteins identified as specific to these pathogens can be detected using various laboratory accepted methods for protein purification and detection (For example Toxin-antitoxin systems, Mycobacterial polyketide synthases, alpha antigen etc.). Chromogenic enzyme assays for a pathogen are also within scope of the invention. Specific metabolites or compounds produced by a pathogen can also be detected (using different laboratory acceptable methods like Mass spectrometry, HPLC-MS, spectrometry-based methods etc.) to ascertain pathogen presence (e.g. Mycobacterial polyketides and lipids). In other embodiments, methods like nucleic acid amplification tests (NAAT), real time PCR, immunoassays for the identified antigens as well as specific staining and microscopy techniques and flow cytometry methods of detecting pathogens are also within scope of this invention. PCR or Restriction Fragment Length Polymorphism (RFLP) based detection of 16S rRNA in order to identify pathogens can also be utilized. In one more embodiment, staining methods can also be utilized to identify a pathogen and establish viability of a pathogen cell (e.g. propidium iodide can be used for identifying dead cells). Cell toxicity assays can also be utilized for toxins based detection of pathogens. Further in case of sporulating bacteria, spore detection assays can also be utilized. In case of culturable bacteria, the viability of pathogens can even be established using culturing methods using selective media followed by methods to detect specific pathogens discussed above. In case of an infection in living beings observation of phenotypic effects like alleviation of infection symptoms is also within scope of this disclosure. The symptoms may vary with type of infection and may be observed by registered medical practitioner or healthcare professional. Any other method of detecting pathogens are also within scope of this disclosure.

According to an embodiment of the disclosure, the DNA extraction module 106 is configured to applying one or more techniques for identification or detection of microbes in a collected sample comprising a sequencing technique, a flow cytometry based methodology, a microscopic examination of the microbes in collected sample, microbial culture of pathogens in vitro, immunoassays, cell toxicity assay, enzymatic, colorimetric or fluorescence assays, assays involving spectroscopic/spectrometric/chromatographic identification and screening of signals from complex microbial populations, The pathogen or microbial characterization data may comprise one or more of sequenced microbial DNA data, a Microscopic imaging data, a Flow cytometry cellular measurement data, a colony count and cellular phenotypic data of microbes grown in in-vitro cultures, immunological data, proteomic/metabolomics data, and a signal intensity data. The sequenced microbial data from sequencer 108 comprises sequences obtained from next generation sequencing platforms comprising one or more of marker genes including 16S rRNA, Whole Genome Shotgun (WGS) sequences, a fragment library based sequences, a mate-pair library or a paired-end library based sequencing technique, or a combination thereof. The sequencing data may also comprise of complete genome sequences of the pathogens obtained within a collected sample. In one embodiment, the taxonomic groups or pathogens within a sample collected can be obtained by amplification of marker genes like 16S rRNA within bacteria. In another embodiment, the taxonomic groups or pathogens within a sample can be obtained by the binning of whole genome sequencing reads into various taxonomic groups using different methods including sequence similarities as well as several methods using supervised and unsupervised classifiers for taxonomic binning of metagenomics sequences.

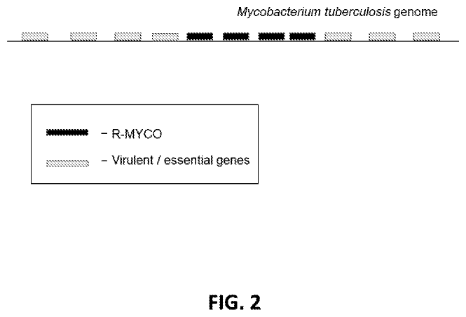

According to an embodiment of the disclosure, the processor 112 comprises the nucleotide repeat sequence identification module 114. The repeat sequence identification module 114 is configured to identify a set of nucleotide sequences in the extracted DNA which occur more than a predefined number of times (refers to the number of occurrences of nucleotide repeat sequence on a genome in a dispersed manner and this number might vary with system and pathogen under consideration) in the genomic sequences of strains of Mycobacterium tuberculosis and are dispersed at distant locations on the genome. The predefined number refers to the number of occurrences of nucleotide repeat sequence on genomic sequences of all pathogenic strains of candidate pathogens in a dispersed manner and this number might vary with system and pathogen under consideration. A minimum of 10 occurrences is required for a nucleotide repeat sequence to be considered. In an example, R-MYCO is identified as shown in schematic representation of FIG. 2. Further, it is important to ensure that the identified nucleotide repeat sequence region is specific to a particular candidate pathogenic genus only (Mycobacterium here) and, on nucleotide sequence based alignment, shows no more than two cross matches with commensals of the other genera or commensals within same genus. Cross match refers to the occurrence of identified nucleotide repeat sequence region more than two times in a genus which is different from the candidate genus in which the nucleotide repeat sequence has been identified as is to be used as a target site.

In addition to that, the identified set of nucleotide sequences are not specific to a single strain of the pathogen. For example, R-MYCO is present in all sequenced strains of Mycobacterium tuberculosis. In most cases, metagenomic samples contain bacteria whose strain level information cannot be obtained. Thus, the method can be utilized to target all pathogens in the given species of the bacteria and is not hindered by the absence of strain level information thereby making it more robust.

Following method can be used for the identification of the repeat sequence region. Conserved nucleotide repeat elements were identified on Mycobacterium tuberculosis genome by taking sequence stretches of predefined length Rn (30-60 in this embodiment), picked from the genome sequence of candidate pathogen or different strains of candidate pathogen Mycobacterium tuberculosis keeping the difference in the start position of consecutive picked nucleotide stretches Rnᵢ₊₁ and Rnᵢ as 5 nucleotides. Predefined length Rn refers to the length of a stretch of nucleotide sequence (picked from the complete nucleotide sequence of a bacterial genome) used as a seed input for local sequence alignment tools. This predefined length may differ depending on the pathogen. In the next step, a reference genome based nucleotide sequence alignment tool is applied in order to align the picked nucleotide sequence stretch with nucleotide sequences corresponding to genomes of all pathogenic strains belonging to the candidate pathogen, genus or specie. Stretches of sequences from the genomic sequences corresponding to strains of Mycobacterium tuberculosis were aligned within the same genome by local alignment (as implemented in PILER software) to find the location of these elements in all sequenced strains of Mycobacterium genomes. Sequence based search utilizing any other sequence alignment or nucleotide repeat finding tools are also within scope of this invention. Sequence based search utilizing BLAST can also be utilized for this purpose. A relaxation of two mismatches was allowed to prevent false positives which could lead to over-prediction of possible targets. If the number of times Rn matches on the genome is greater than the predefined threshold (refers to the number of occurrences of nucleotide repeat sequence on a genome in a dispersed manner and this number might vary with system and pathogen under consideration. The number of occurrences of the nucleotide repeat sequence is 15 in this embodiment, the sequence stretch is termed as R-MYCO. It should be noted that these R-MYCO sequences are dispersed across the genome in distant locations so that multiple regions of the pathogen genome can be targeted simultaneously. Although, the number of occurrences of the nucleotide repeat sequence might vary in different pathogens, a minimum of 10 occurrences is required for a nucleotide repeat sequence to be considered as a target sequence The dispersed nucleotide sequences at distant locations on the genome refers to stretches of nucleotide sequences which occur across the genome with a distance of predefined number of base pairs between them In one embodiment used in this disclosure the predefined number refers to a separation of >10000 base pairs between two nucleotide repeat sequences. If the number of times Rₙ matches on the genomic sequences of strains of candidate pathogen genome/genomes is greater than the predefined threshold with a minimum value of 10, the sequence stretch is termed as target nucleotide repeat sequence. The nucleotide repeat sequences which are conserved across all genome sequences corresponding to strains of a candidate pathogen or genus would indicate the said conserved sites. Any other method of identification of conserved sites is also within the scope of this disclosure.

According to an embodiment of the disclosure, the memory 110 further includes the neighborhood gene identification module 116. The neighborhood gene identification module 116 is configured to identify a set of neighborhood genes present upstream and downstream (on the nucleotide sequence on the genome of the candidate pathogen) of the set of nucleotide repeat sequences corresponding to Mycobacterium tuberculosis. On each Mycobacterium genome where nucleotide repeat elements or their reverse complement occur, 10 flanking genes both upstream and downstream were found on each strand (+ and −) of DNA. The number of flanking genes considered may vary with the system.

According to an embodiment of the disclosure, the system 100 further includes the annotation module 118. The annotation module 118 categorizes or annotates the set of neighborhood genes based on their functional roles in the pathogen. Functional annotation of these genes was performed using HMM search with PFAM as the database. In other embodiments, databases like CDD, SMART etc. can be utilized. The use of any other methods such as PSSM, BLAST etc. is well within the scope of the disclosure.

These dispersed repeat sequences RMYCO can be used as targets which can be further extended to target multiple flanking genes (which includes virulence and survival genes) simultaneously at distant multiple locations and carry out changes like but not limited to gene silencing, gene recombination, gene substitution with a new function etc.

Functional categorization of these genes on the basis of pathways they are involved in was carried out using literature mining. The broad categories have been discussed in Table 1.

TABLE 1
Summary of proteins in vicinity conserved sequence R-MYCO
in Mycobacterium tuberculosis genome.
Essential Proteins

| | | |
|---|---|---|
| Metabolism | Lipase enzymes | Involved in metabolizing |
| | | variety of fatty acids |
| | Fad proteins | Fatty acid metabolism |
| | Glg gene cluster | Utilization of glucose |
| Transcription/ | Ribosomal protein L25 | Ribosomal assembly |
| Translation | Transcriptional regulators | Multiple gene clusters |
| Cell wall | D-Alanine ligase Muramic | Peptidoglycan layer |
| biosynthesis | acid biosynthesis | |

Virulence/Pathogenic proteins

| | | |
|---|---|---|
| Virulence | PcaA, mma gene cluster | Mycolic acid synthesis |
| | Fasciclin | Adhesin |
| | Chalcone synthase | Polyketide biosynthesis |
| | PKS7 | |
| | Other PKS cluster | |
| | Maz and Vap toxins | Toxin antitoxin systems |
| | Guanylate cyclase | c-di-GMP formation |
| | ESAT-6 | Virulent protein |
| | DisA | cAMP biosynthesis |
| | PE-PGRS and PPE | Pathogenesis |
| Stress response | Ribonuclease | RNAprocessing |
| | Clp protease | Stress response |
| | Helicases Ruv and Uvr | Repair machinery |

According to an embodiment of the disclosure, the system 100 further includes the testing module 120 and the administration module 122. The testing module 120 is configured to check the presence of secondary structure formation such as hairpin loop structures in the identified set of nucleotide repeat sequence. Depending on the presence of the secondary structure, the administration module 122 is configured to administer an engineered polynucleotide construct to treat the pathogenic infection, wherein the engineered polynucleotide construct is comprising: one or more of the first and the second set of nucleotide repeat sequences with multiple copies at dispersed locations on the candidate pathogen genomes of one or more of the pathogenic strains of Mycobacterium tuberculosis, wherein the first set of nucleotide repeat sequences comprises a Sequence ID 001 or reverse complement of the sequence ID 001, a first enzyme capable of nicking and cleaving the identified set of nucleotide sequences, and a second enzyme capable of removal of a set of neighborhood genes flanking the set of nucleotide repeat sequences. The engineered polynucleotide construct works in such a way that it targets multiple regions in the genome simultaneously.

In an embodiment the engineered polynucleotide construct may comprise of an engineered circular DNA comprising of an origin of replication. Further the engineered polynucleotide construct may comprise of regulatory elements including a promoter sequence, ribosomal binding site, start codon, a cassette comprising of first and second enzyme flanking the nucleotide repeat sequence or its reverse complement of the nucleotide repeat sequence R-MYCO cloned into the system, stop codons and transcription terminator. The promoter sequence may depend on the pathogen being targeted as well as the regulation required to express the components of the engineered polynucleotide construct at a specific targeted site (for example, within a living being or an infected area). The engineered polynucleotide construct may also be equipped to create a polyA tail in mRNA to stabilize the sequence. The poly A tail refers to a stretch of polynucleotide Adenine nucleotides at the 3′ end of mRNA. In one embodiment, the first and second enzyme can be nickase and exonuclease cloned in any order. The target R-MYCO within the pathogen genome can be recognized and bound by the reverse complement sequence and the complex thus formed can be nicked by the nickase enzyme. The exonuclease can then cut the duplex formed as well as flanking genes once it recognizes a nick. In another embodiment, the enzymes can be cas9 sequences (may be obtained from Streptococcus pyogenes) flanking the RMYCO or flanking the reverse complement of R-MYCO which can both act as sgRNA (single guide RNA) for the obtained CRISPR-Cas (Clustered Regularly Interspaced Short Palindromic Repeats) system. The reverse complement of target nucleotide repeat sequence is obtained by interchanging letters A and T and interchanging letters C and G between target and complement sequences. The reverse complement refers to the sequence corresponding to the identified nucleotide repeat sequence in the opposite strand of DNA. The R-MYCO or its reverse complement is recognized by the reverse complement sequence or the target sequence on the engineered polynucleotide construct and the complex formed by the binding of R-MYCO sequence to its reverse complement. The cas9 may then act as an endonuclease and cut the nick and flanking sequences. The nucleotide repeat sequence can be targeted by delivering an engineered polynucleotide construct using a bacterial, plasmid or a viral vector to the target bacterial cell. In one embodiment the composition may comprise of: the first element comprising a polynucleotide sequence of CRISPR-Cas system wherein the polynucleotide sequence may comprise a nucleotide repeat sequence (identified repeat or its reverse complement) called a guide sequence capable of hybridizing to target sequence (nucleotide repeat sequence on pathogen), a tracr sequence and a tracr mate sequence. The second element may comprise of CRISPR enzyme coding sequences like CAS enzymes. It should be noted that in all these embodiments RMYCO sequences can be cloned within same polynucleotide sequence along with a bacterial or viral vector and the other features mentioned above to target more than one pathogen using the same compact construct. Any other construct cassette that may bring about the recognition of the RMYCO sequences in bacterial genomes and subsequent nicking and chopping of RMYCO sequences and the flanking genes is within the scope of this invention.

In another embodiment, in addition to the above mentioned features, if bacterial conjugation is to be used as a construct delivery method, the engineered polynucleotide construct may comprise of a relaxase, coding sequences for structural proteins (e.g. pili) and those for regulatory proteins for conjugation. It should be noted that in both embodiments multiple R-MYCO sequences can be cloned to target more than one pathogen using the same compact construct. Any other construct cassette that may bring about the recognition of the R-MYCO and subsequent chopping of R-MYCO and the flanking genes is within the scope of this invention. These polynucleotides comprising the nucleotide repeat sequence, the genes encoding enzymes and the other features discussed above can be inserted into laboratory acceptable vectors which allow insertion of external DNA fragments; In one embodiment construct may be carried by vectors like plasmid or phage based cloning vectors. The regulatory elements can be designed according to information available for the pathogen being targeted.

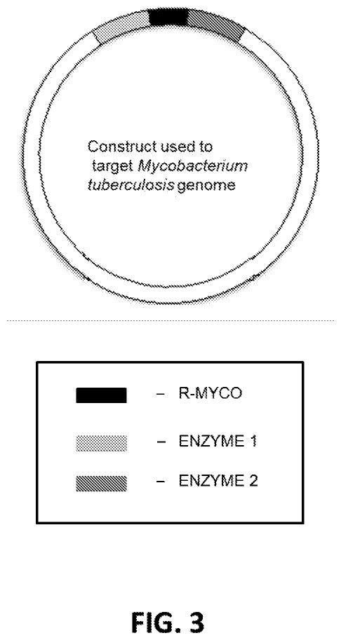

In one embodiment, the engineered polynucleotide construct may contain an enzyme 1, enzyme 2, identified nucleotide target sequence (R-MYCO) as shown in FIG. 3. One of the enzyme 1 or enzyme 2 can be the nicking enzyme while the other will constitute nucleotide cleaving enzymes such as nuclease, exo-nuclease etc. Other enzymes with similar activities are also within scope of the invention.

Depending on the result of testing module 120, there could be two cases as follows:

Case I: If the identified nucleotide repeat sequences are found to be palindromic the following three strategies may be used.

Strategy I includes handling hairpin loops which hinders DNA transcription by stalling the RNA polymerase enzyme thereby down-regulating the flanking gene expression. In an embodiment, the strategy would involve use of the identified nucleotide repeat sequences as target and inserting a strong palindromic sequence to ensure the down-regulation of transcription of flanking genes

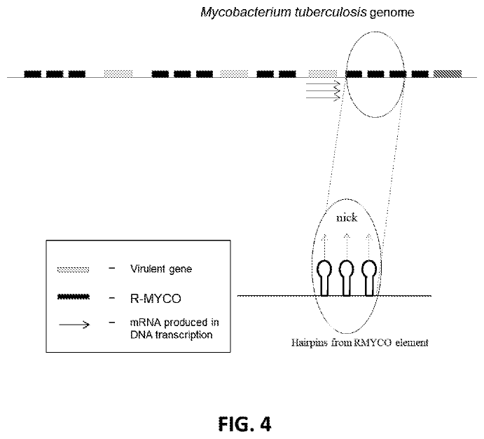

Strategy II involves handling hairpin loops formed in the mRNA which could be involved in prevention of the early decay of mRNA thereby promoting the expression of important bacterial genes. In an embodiment, the strategy may include use of the identified repeat sequences as target to nick the pathogen genome at multiple locations and cleave the flanking genes. In an example, a schematic representation of the Mycobacterium tuberculosis genome showing nick of Hairpins from R-MYCO element is shown in FIG. 4.

Strategy III is utilized if the identified nucleotide repeat sequences is found to be a transcription terminator and is followed by a polyA tail. In an embodiment, the identified nucleotide repeat sequence is used as target and a strong palindromic sequence is inserted to ensure that the transcriptional termination of the flanking genes occur and these genes are down-regulated in the pathogen.

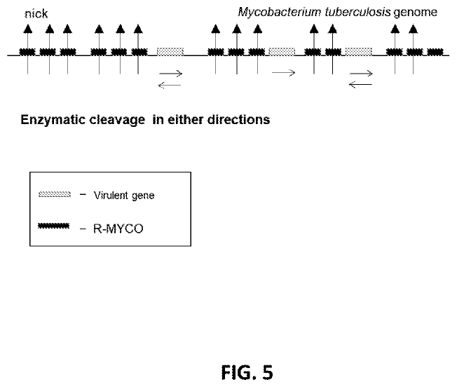

Case II: If the identified nucleotide repeat sequences are not found to be palindromic, the identified nucleotide repeat sequences are used as target to nick the pathogen genome at multiple locations and cleave the flanking genes. A schematic representation of Mycobacterium tuberculosis genome showing enzymatic cleavage in either directions is shown in FIG. 5.

In the present embodiment, the R-MYCO sequence is used as an example. If the R-MYCO is shown to inhibit flanking genes by stalling RNA polymerase: R-MYCO can be used as a target and a strong palindromic sequence is inserted to ensure the down-regulation of transcription of flanking genes. Palindromic sequences in a transcription bubble form hairpin loops which hinders DNA transcription by stalling the RNA polymerase enzyme thereby down-regulating the flanking gene expression.

If the R-MYCO is shown to promote flanking genes by increasing mRNA stability: R-MYCO can be used as target to nick the pathogen genome at multiple locations and the flanking genes are cleaved. Hairpin loops formed in the mRNA could be involved in prevention of the early decay of mRNA, and cleaving these sequences impedes the expression of the flanking genes.

In the present embodiment, the R-MYCO sequences, are palindromic and may form a hairpin loop structure indicating their role in regulation of transcription. These loops may either form at DNA level or at the ends of their mRNA during DNA transcription. This hairpin loop in the mRNA could be involved in prevention of the early decay of mRNA, resulting in higher protein formation of the virulence genes which are in the vicinity of these palindromic elements. Reduction in pathogenicity can be achieved by decreasing the stability of mRNA corresponding to these virulent genes which can be attained by removing the hairpin loops. If hairpin loop formation takes place at DNA level it might regulate DNA supercoiling and concatenation. The hairpin loop is not followed by a polyA tail or an AT rich region indicating it might not be working as a transcription terminator also.

The administration module 122 can use any pharmaceutically acceptable method of carrying the engineered polynucleotide construct to target the conserved sequences in a pathogen genome. In different embodiments the utility can be, but not limited to oral medicine, topical creams, nasal administration, aerosol sprays, injectable cocktail etc.

In an embodiment, the engineered polynucleotide construct can be administered to the infected site (either living beings or environmental site) through targeted construct delivery methods such as the use of targeted liposomes (wherein, the liposome is tagged on the external surface with molecules that may be specific and functionally important to the candidate genus and the tagged liposome can be used to transfer the engineered polynucleotide construct into the pathogen), targeted nanoparticles (wherein, a targeting molecule that is specific to the candidate genus can be attached to the nanoparticle (like but not limited to Ag or Au nanoparticle) along with the engineered polynucleotide construct, thereby allowing the tagged nanoparticle to release the engineered polynucleotide construct into the pathogen), phage based delivery method (wherein, the engineered polynucleotide construct can be placed within the phage infecting the candidate genus thereby transferring the engineered polynucleotide construct into pathogen) and bacterial conjugation (wherein, the engineered polynucleotide construct can be placed in other bacteria that can conjugate with the candidate genus and the engineered polynucleotide construct can be transferred to the pathogen through natural conjugation method) etc. In an embodiment, the lipid constitution of the membrane for the targeted liposome can be modified to target specific set of bacteria.

In another embodiment, immunoliposomes can be created with specific antibodies towards ligands of specific pathogen (for example, antibodies against concanavalin A for targeting extracellular matrix of biofilms). The lipid bilayer can be made sensitive to the toxins or other virulence factors of the pathogen in order to release the engineered polynucleotide construct only in infected areas where toxins are present.

In another embodiment, the engineered polynucleotide construct can also be administered to the infected site through non-targeted construct delivery methods such as the use of non-targeted nanoparticles (wherein, nanoparticles can form cages that can hold the engineered polynucleotide construct which are then released into the pathogen), non-targeted liposomes (wherein, the liposomes are phospholipid capsules which can be used to hold the engineered polynucleotide construct that can then merge with the pathogen cell membrane to release the engineered polynucleotide construct inside the pathogen) etc. In an embodiment, attenuated bacteria can also be used to deliver nanoparticles into tissue spaces where they can be released to act upon actual site of infection (as shown in creation of NanoBEADS in a study where Salmonella was used to deliver nanoparticles containing a drug to deep tissues). In another example, minicells produced by bacteria can also be used to package the engineered polynucleotide construct and deliver it to specific areas in the infected site. In another embodiment, these delivery methods can be used to target the engineered polynucleotide construct to infected surfaces also. Any other laboratory accepted method of administration of the engineered polynucleotide construct to the infected site is within the scope of this disclosure.

According to an embodiment of the disclosure, the efficacy module 124 is used to assess the efficacy of the treatment methodology described in this disclosure. The efficacy module 124 comprises of any laboratory acceptable methods of detecting presence of pathogens present at the infected site. In one embodiment, presence of viable living cells can be detected by utilizing presence of bacterial mRNA which has a short half-life and will not exist once the cells are dead. This mRNA based method may involve identifying antigen/protein specific for the pathogen which can be utilized as a marker for that pathogen and produced by the pathogen in abundance and the corresponding gene on the pathogen genome can be obtained (alpha antigen in Mycobacterium tuberculosis etc). The mRNA corresponding to expression of these genes can be detected using techniques like but not limited to polymerase chain reaction (RT-PCR) assays or reverse transcriptase strand displacement amplification (RT-SDA) assays. In another embodiment, expression of proteins identified as specific to these pathogens can be detected using various laboratory accepted methods for protein purification and detection (For example Toxin-antitoxin systems, Mycobacterial polyketide synthases, alpha antigen etc. etc.). Chromogenic enzyme assays for a pathogen are also within scope of the invention. Specific metabolites or compounds produced by a pathogen can also be detected (using different laboratory acceptable methods like Mass spectrometry, HPLC-MS, spectrometry-based methods etc.) to ascertain pathogen presence (e.g. Mycobacterial polyketides and lipids). In other embodiments, methods like nucleic acid amplification tests (NAAT), real time PCR, immunoassays for the identified antigens as well as specific staining and microscopy techniques and flow cytometry methods of detecting pathogens are also within scope of this invention. PCR or Restriction Fragment Length Polymorphism (RFLP) based detection of 16S rRNA in order to identify pathogens can also be utilized. In one more embodiment, staining methods can also be utilized to identify a pathogen and establish viability of a pathogen cell (e.g. propidium iodide can be used for identifying dead cells). Cell toxicity assays can also be utilized for toxins based detection of pathogens. Further in case of sporulating bacteria, spore detection assays can also be utilized. In case of culturable bacteria, the viability of pathogens can even be established using culturing methods based on selective media followed by methods to detect specific pathogens discussed above. In case of an infection in living beings observation of phenotypic effects like alleviation of infection symptoms is also within scope of this disclosure. The symptoms may vary with type of infection and may be observed by registered medical practitioner or healthcare professional. Any other method of detecting pathogens are also within scope of this disclosure. In case pathogen presence is detected, the engineered polynucleotide construct can be administered again using administration module 120 and repeated till pathogen is eliminated.

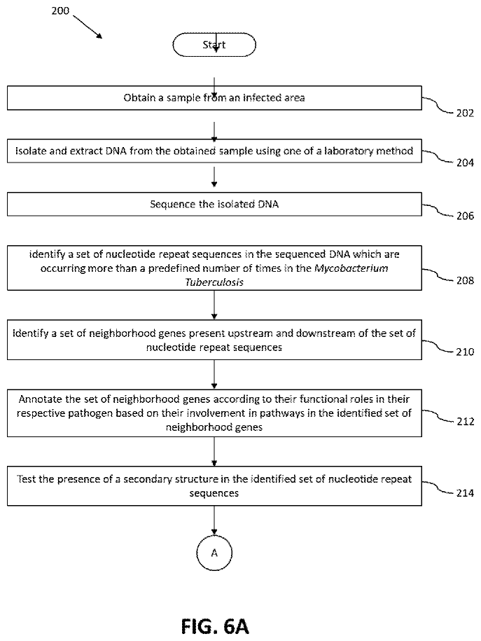

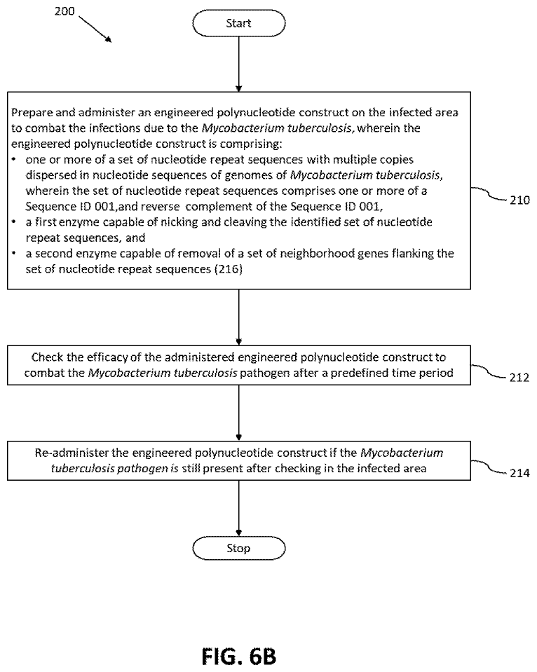

In operation, a flowchart 200 illustrating the steps involved for combating infections due to Mycobacterium tuberculosis can be shown in FIG. 6A-6B. Initially at 202, a sample is obtained from an area infected from the pathogen Mycobacterium tuberculosis. At step 204, DNA is extracted from the sample using the pathogen detection and DNA extraction module 106 which is configured for pathogen detection. At step 206, the extracted DNA is sequenced using the sequencer 108. In the next step 208, the set of nucleotide repeat sequences in the extracted DNA is identified which occur more than a predefined number of times (refers to the number of occurrences of nucleotide repeat sequence on a genome in a dispersed manner and this number might vary with system and pathogen under consideration where the minimum value of predefined number is 10) in the Mycobacterium tuberculosis. In an example, the identified set of nucleotide sequences correspond to R-MYCO. In addition to that, the identified set of nucleotide repeat sequences are not specific to a single strain of the pathogen. In addition to that, the identified set of nucleotide repeat sequences are not specific to a single strain of the pathogen. In step 210, the set of neighborhood genes present upstream and downstream of the set of nucleotide repeat sequences was identified.

In step 212, the set of neighborhood genes is categorized or annotated according to functional roles of each of neighborhood gene in the Mycobacterium tuberculosis. At step 214, the presence of the secondary structure is tested in the set of nucleotide repeat sequences. The set of nucleotide repeat sequences may be palindromic in nature which may result in the formation of hairpin loops.

At step 216, an engineered polynucleotide construct is prepared and administered on the infected area to combat the infections due to Mycobacterium tuberculosis if palindromic formation is not there, wherein the engineered polynucleotide construct is comprising:
one or more of the set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001
a first enzyme capable of nicking and cleaving the identified set of nucleotide repeat sequences, and
a second enzyme capable of removal of a set of neighborhood genes flanking the set of nucleotide repeat sequences;

The administration of construct aims at targeting the set of identified nucleotide repeats and removal of flanking genes on genomes of pathogen infecting the area. The engineered polynucleotide construct works in such a way that it targets multiple regions in the pathogenic genome simultaneously. At step 218, the efficacy of the administration module is assessed and in case Mycobacterium tuberculosis pathogen presence is detected at the site, administration module can be utilized repetitively till Mycobacterium tuberculosis pathogen is eliminated from the site. And finally at step 220, the engineered polynucleotide construct is re-administered if the Mycobacterium tuberculosis is still present after checking using efficacy module in the infected area.

According to an embodiment of the disclosure, the system 100 can also be used in combination with various other known methods to effectively treat the pathogenic infection due to Mycobacterium tuberculosis. In an example, the method 200 can be used as preventive method. The method can be used in combination with various other antibacterial agents. One implementation would be the use of quorum quenchers along with the engineered polynucleotide construct to tackle the biofilm formation in hospital surfaces. In another example, the method may be used as a therapeutic measure. The method may be used in combination with various other antimicrobial methods. One implementation would be to use the method along with antibiotics and vaccines against essential proteins for therapeutic purposes.

According to an embodiment of the disclosure, the system 100 for combating infections due to Mycobacterium tuberculosis can also be explained with the help of following example for Mycobacterium tuberculosis.

Nucleotide repeat sequences were identified on sequenced Mycobacterium tuberculosis genomes by taking a sequence stretch of predefined length Rn and searching across the genome for similar sequence stretches as taught by several alignment software. Nucleotide repeat sequence elements were identified to be the sequence:

CAGAC(G|A)C(A|G)NAANCNCNNNNNNN₍₁₀₋₂₅₎NNGNGNTTN
(T|C)G(C|T)GTCTG

On further analysis, it was observed that these conserved stretches (as discussed above) are found in the vicinity of highly virulent and, certain essential genes of Mycobacterium as provided in TABLE 1. Results of sequence similarity analysis (using BLAST in this embodiment) revealed that this sequence doesn't show any significant sequence similarity in any other bacterial genus and on the human host genome reducing the possibility of a cross-reactivity. Hence, these elements are ideal candidates for targeting pathogenic Mycobacterium tuberculosis.

On each Mycobacterium tuberculosis genome where nucleotide repeat elements R-MYCO occur, 10 flanking genes both upstream and downstream were found on each strand (+ and −) of DNA. Functional annotation of these genes was performed using HMM search with PFAM as the database. Functional categorization of these genes on the basis of pathways they are involved in was carried out using literature mining. The broad categories have been discussed in Tables 1.

In the present example, the R-MYCO sequence palindromic and may form a hairpin loop structure with a significant free energy value. They also seem to not be either canonical or non-canonical Mycobacterium terminators as seen by the absence of poly-A tail. If the R-MYCO is shown to inhibit flanking genes by stalling the RNA polymerase: R-MYCO can be used as target and a strong palindromic sequence is inserted to ensure the down-regulation of transcription of flanking genes. Palindromic sequences in a transcription bubble form hairpin loops which hinders DNA transcription by stalling the RNA polymerase enzyme thereby down-regulating the flanking gene expression. If the R-MYCO is shown to promote flanking genes by increasing mRNA stability: R-MYCO can be used as target to nick the pathogen genome at multiple locations and the flanking genes are cleaved. Hairpin loops formed in the mRNA could be involved in prevention of the early decay of mRNA, and cleaving these genes impedes the expression of the flanking genes.

Depending on the presence of the hairpin loop structure, one of the strategies mentioned above can be used to combat infections due to Mycobacterium tuberculosis.

The embodiments of present disclosure herein provides a method and system for combating infections due to Mycobacterium tuberculosis.

Sequences and their reverse complements have been disclosed Sequence 001: Mycobacterium tuberculosis:

CAGAC(G|A)C(A|G)NAANCNCNNNNNNN(10-25)
NNGNGNTTN(T|C)G(C|T)G TCTG

where N refers to any nucleotide out of A, T, G and C and numeric values in subscripts indicate the range of the number of times a nucleotide or a set of nucleotides is repeated in the sequence.
Following is the number of occurrences and locations of repeats in the strains from Mycobacterium Tuberculosis is as follows. Due the large number of available strain, only few well characterized ones with maximum occurrences of Sequence 001 corresponding to R-MYCO are provided below:

### Mycobacterium_tuberculosis_H37Rv_-_GCA_000277735.2_ASM27773v2

Number of occurrences: 41
[(234449, 234499), (279539, 279589), (456207, 456257), (459393, 459443), (558825, 558875), (663397, 663447), (736240, 736290), (767332, 767382), (829714, 829764), (842310, 842360), (1000779, 1000829), (1064061, 1064111), (1133273, 1133323), (1182330, 1182380), (1205248, 1205297), (1224319, 1224369), (1357075, 1357125), (1363449, 1363499), (1488095, 1488145), (1568057, 1568107), (1828811, 1828861), (1872581, 1872631), (2075544, 2075594), (2317115, 2317166), (2466978, 2467028), (2522185, 2522235), (2700483, 2700533), (2907766, 2907816), (3059805, 3059855), (3075393, 3075443), (3104993, 3105043), (3348495, 3348545), (3590632, 3590682), (3628088, 3628138), (3707747, 3707797), (3724729, 3724779), (3804038, 3804088), (3909942, 3909992), (4008838, 4008888), (4021577, 4021627), (4246708, 4246758)]

### Mycobacterium_tuberculosis_H37Ra_-_GCA_001938725.1_ASM193872v1

Number of occurrences: 41
[(235810, 235860), (280900, 280950), (457568, 457618), (460754, 460804), (560186, 560236), (664758, 664808), (737597, 737647), (768689, 768739), (831071, 831121), (843667, 843717), (1002137, 1002187), (1065419, 1065469), (1134631, 1134681), (1183688, 1183738), (1206606, 1206655), (1225677, 1225727), (1358433, 1358483), (1364807, 1364857), (1489453, 1489503), (1569415, 1569465), (1830376, 1830426), (1874146, 1874196), (2086634, 2086684), (2328205, 2328256), (2478068, 2478118), (2533275, 2533325), (2713637, 2713687), (2920920, 2920970), (3072959, 3073009), (3088547, 3088597), (3118147, 3118197), (3361826, 3361876), (3602605, 3602655), (3640061, 3640111), (3719720, 3719770), (3736702, 3736752), (3817739, 3817789), (3923643, 3923693), (4023238, 4023288), (4035977, 4036027), (4261108, 4261158)]

### Mycobacterium_tuberculosis_BT2_-_GCA_000572155.1_ASM57215v1

Number of occurrences: 43
[(232771, 232821), (277861, 277911), (454536, 454586), (457722, 457772), (557152, 557202), (661852, 661902), (734691, 734741), (766336, 766386), (828826, 828876), (841295, 841345), (999483, 999533), (1062762, 1062812), (1133345, 1133395), (1182402, 1182452), (1205485, 1205534), (1224552, 1224602), (1358666, 1358716), (1365040, 1365090), (1491207, 1491257), (1572492, 1572542), (1825762, 1825812), (1869553, 1869603), (2062456, 2062506), (2303229, 2303280), (2338220, 2338270), (2452043, 2452093), (2507250, 2507300), (2684256, 2684306), (2889953, 2890003), (3041092, 3041142), (3056680, 3056730), (3086280, 3086330), (3325611, 3325661), (3401251, 3401301), (3572595, 3572645), (3610052, 3610102), (3689460, 3689510), (3710244, 3710294), (3789733, 3789783), (3899158, 3899208), (3998590, 3998640), (4011329, 4011379), (4236838, 4236888)]

### Mycobacterium_tuberculosis_CCDC5079_-_GCA_000400615.1_ASM40061v1

Number of occurrences: 43
[(232716, 232766), (277806, 277856), (454532, 454582), (457718, 457768), (557157, 557207), (661789, 661839), (734627, 734677), (766252, 766302), (828688, 828738), (841157, 841207), (999347, 999397), (1062626, 1062676), (1131836, 1131886), (1180893, 1180943), (1203976, 1204025), (1223043, 1223093), (1357158, 1357208), (1363532, 1363582), (1489700, 1489750), (1569623, 1569673), (1822882, 1822932), (1866672, 1866722), (2060820, 2060870), (2317012, 2317063), (2351885, 2351935), (2466955, 2467005), (2522162, 2522212), (2696583, 2696633), (2902289, 2902339), (3053221, 3053271), (3070168, 3070218), (3099768, 3099818), (3337797, 3337847), (3413436, 3413486), (3583419, 3583469), (3620876, 3620926), (3700284, 3700334), (3721068, 3721118), (3802198, 3802248), (3911332, 3911382), (4010758, 4010808), (4023497, 4023547), (4249229, 4249279)]

### Mycobacterium_tuberculosis_CCDC5180_-_GCA_000572195.1_ASM57219v1

Number of occurrences: 43
[(232715, 232765), (277805, 277855), (454532, 454582), (457718, 457768), (557148, 557198), (661668, 661718), (734507, 734557), (766152, 766202), (828642, 828692), (841111, 841161), (999309, 999359), (1062588, 1062638), (1131696, 1131746), (1180754, 1180804), (1203837, 1203886), (1221329, 1221379), (1355443, 1355493), (1361817, 1361867), (1487984, 1488034), (1567911, 1567961), (1821217, 1821267), (1865008, 1865058), (2059272, 2059322), (2314031, 2314082), (2349020, 2349070), (2462616, 2462666), (2517823, 2517873), (2694829, 2694879), (2900534, 2900584), (3051466, 3051516), (3067054, 3067104), (3096654, 3096704), (3334682, 3334732), (3412210, 3412260), (3583483, 3583533), (3620940, 3620990), (3700350, 3700400), (3721134, 3721184), (3802352, 3802402), (3911371, 3911421), (4010839, 4010889), (4023578, 4023628), (4249251, 4249301)]

### Mycobacterium_tuberculosis_CTRI-2_-_GCA_000224435.1_ASM22443v1

Number of occurrences: 43
[(234205, 234255), (279295, 279345), (455924, 455974), (459110, 459160), (558542, 558592), (663058, 663108), (735892, 735942), (767517, 767567), (830114, 830164), (842789, 842839), (1001192, 1001242), (1064472, 1064522), (1133695, 1133745), (1182753, 1182803), (1205671, 1205720), (1224676, 1224726), (1357432, 1357482), (1363806, 1363856), (1488396, 1488446), (1566992, 1567042), (1820055, 1820105), (1863825, 1863875), (2069246, 2069296), (2317032, 2317083), (2352008, 2352058), (2464046, 2464096), (2519253, 2519303), (2696759, 2696809), (2902658, 2902708), (3053496, 3053546), (3069084, 3069134), (3098684, 3098734), (3344940, 3344990), (3419696, 3419746), (3585063, 3585113), (3622532, 3622582), (3701953, 3702003), (3723020, 3723070), (3800914, 3800964), (3900652, 3900702), (3994124, 3994174), (4006863, 4006913), (4233519, 4233569)]

### Mycobacterium_tuberculosis_F11_-_GCA_000016925.1_ASM1692v1

Number of occurrences 43
[(234767, 234817), (279857, 279907), (459374, 459424), (462560, 462610), (561991, 562041), (666622, 666672), (739465, 739515), (771090, 771140), (833580, 833630), (846176, 846226), (1004601, 1004651), (1067880, 1067930), (1137037, 1137087), (1186095, 1186145), (1209013, 1209062), (1228081, 1228131), (1360833, 1360883), (1367208, 1367258), (1494888, 1494937), (1572392, 1572442), (1824195, 1824245), (1867964, 1868014), (2083733, 2083783), (2331719, 2331770), (2368054, 2368104), (2479116, 2479166), (2534323, 2534373), (2715126, 2715176), (2921073, 2921123), (3071750, 3071800), (3087338, 3087388), (3116938, 3116988), (3360194, 3360244), (3434950, 3435000), (3602400, 3602450), (3639869, 3639919), (3719233, 3719283), (3736885, 3736935), (3814975, 3815025), (3922116, 3922166), (4021440, 4021490), (4034179, 4034229), (4259531, 4259581)]

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address unresolved problem of antimicrobial resistance as can be observed in multi-drug resistant and extensively drug resistant pathogens comprising Mycobacterium Tuberculosis. The embodiment provides a system and method to combat infections due to Mycobacterium Tuberculosis.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

1. A method for combating infections due to Mycobacterium Tuberculosis, the method comprising:
obtaining a sample from an infected area;
isolating and extracting DNA from the obtained sample using one of a laboratory method;
sequencing the isolated DNA using a sequencer;
identifying a set of nucleotide repeat sequences in the sequenced DNA which are occurring more than a predefined number of times in the Mycobacterium Tuberculosis;
identifying a set of neighborhood genes present upstream and downstream of the set of nucleotide repeat sequences;
annotating the set of neighborhood genes according to their functional roles in their respective pathogen based on their involvement in pathways in the identified set of neighborhood genes;
testing the presence of a secondary structure in the identified set of nucleotide repeat sequences;
preparing and administering an engineered polynucleotide construct on the infected area to combat the infections due to the Mycobacterium tuberculosis, wherein the engineered polynucleotide construct is comprising:
one or more of a set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001,
a first enzyme capable of nicking and cleaving the identified set of nucleotide repeat sequences, and
a second enzyme capable of removal of a set of neighborhood genes flanking the set of nucleotide repeat sequences;
checking the efficacy of the administered engineered polynucleotide construct to combat the Mycobacterium tuberculosis after a predefined time period; and
re-administering the engineered polynucleotide construct if the Mycobacterium tuberculosis are still present in the infected area post administering.

2. The method according to claim 1 wherein the samples obtained from infected area is one or more of fecal matter, blood, urine, tissue biopsy, hospital surfaces or environmental samples, and wherein the DNA isolation and extraction methods comprise laboratory standardized protocols including DNA isolation and extraction kits.

3. (canceled)

4. The method according to claim 1 wherein the plurality of pathogen detection method comprises one or more of:
a sequencing technique,
a flow cytometry based methodology,
a microscopic examination of the microbes in collected sample,
a microbial culture of pathogens in vitro, immunoassays, cell toxicity assay, enzymatic, colorimetric or fluorescence assays, assays involving spectroscopic/spectrometric/chromatographic identification and screening of signals from complex microbial populations.

5. The method according to claim 1, wherein the pathogen detection comprise one or more of sequenced microbial DNA data, a microscopic imaging data, a flow cytometry cellular measurement data, a colony count and cellular phenotypic data of microbes grown in in-vitro cultures, immunological data, proteomic/metabolomics data, and a signal intensity data.

6. The method according to claim 1 further comprising sequenced microbial data, wherein the sequenced microbial data comprises sequences obtained from sequencing platforms comprising sequences of marker genes including 16S rRNA, Whole Genome Shotgun (WGS) sequences, sequences obtained from a fragment library, sequences from a mate-pair library or a paired-end library based sequencing technique, a complete sequence of pathogen genome or a combination thereof, wherein, the pathogen detection in the sample depend on identification of taxonomic groups from these sequences.

7. The method according to claim 1, wherein the polynucleotides are inserted into vectors which allow insertion of external DNA fragments, wherein the engineered polynucleotide construct is carried by plasmid or phage based cloning vectors, wherein the engineered polynucleotide construct further comprises bacteria specific promoter sequence, a terminator sequence, a stretch of Thymine nucleotides which is transcribed into a polyA tail for stabilizing the mRNAs transcripts corresponding to each enzyme, wherein the promoters and terminators specific to candidate bacteria can be utilized in the engineered polynucleotide construct.

8. The method according to claim 1 wherein the engineered polynucleotide construct comprises of a CRISPR-Cas system, comprising:
a CRISPR enzyme,
a guide sequence capable of hybridizing to the identified target nucleotide repeat sequence within the pathogen genome,
a tracr mate sequence, and
a tracr sequence,
wherein the guide sequence, the tracr mate and the tracr sequences are linked to one regulatory element of the engineered polynucleotide construct while the CRISPR enzyme is linked to another regulatory module within the vector.

9. The method according to claim 1, wherein the engineered polynucleotide construct is administered using one or more of following delivery methods:
liposome encompassing the engineered polynucleotide construct,
targeted liposome with a ligand specific to the target pathogen on the external surface and encompassing the engineered polynucleotide construct to be administered,
using nanoparticles like Ag and Au,
gene guns or micro-projectiles where the engineered polynucleotide construct is adsorbed or covalently linked to heavy metals which carry it to different bacterial cells, or
bacterial conjugation methods and bacteriophage specific to the targeted pathogen.

10. The method according to claim 1, wherein the first enzyme is a nicking enzyme and the second enzyme is a cleaving enzyme.

11. The method according to claim 1, wherein the set of nucleotide repeat sequences corresponding to one or more than one strain of the Mycobacterium tuberculosis pathogen or candidate genus or species, wherein the set of nucleotide repeat sequences are found in multiple copies at distant locations on the genomes of all pathogenic strains of candidate genus or specie and these nucleotide repeat sequences do not show more than two nucleotide sequence similarity based match to genome sequences corresponding to genera or species other than the genome sequences of pathogens belonging to the candidate genus or species or with genomes of commensal strains within the candidate genus or specie; wherein distant locations refer to distance of greater than 10000 nucleotide base pairs.

12. The method according to claim 1 further comprising the step identifying the set of nucleotide sequences comprises:
selecting a nucleotide sequence stretches of a predefined length Rₙ from the genomes of strains of candidate pathogen with a difference in the start position of two consecutive nucleotide stretches Rₙᵢ₊₁ and Rₙᵢ as 5 nucleotides, wherein the predefined length refers to the length of a stretch of nucleotide sequence picked from the complete nucleotide sequence of a bacterial genome, used as a seed input for local sequence alignment tools,
aligning a stretch of sequences within the genome of candidate pathogen genus/specie or with genomes of all strains of the candidate pathogen genus/specie Mycobacterium tuberculosis, and
identifying the set of nucleotide repeat sequences, repeating more than 10 times at distant locations on the bacterial genome as the set of nucleotide repeat sequences, wherein the set of nucleotide repeat sequences with repeats comprising of one or more of a Sequence ID 001 or a complement of the Sequence ID 001.

13. The method according to claim 1, wherein the identified nucleotide repeat sequences are in genomic neighborhood of or flanking the genes encoding proteins with essential functions within a pathogen genome, wherein the genomic neighborhood refers to regions lying within a predefined number of genes to the selected nucleotide repeat sequence or the reverse complement of the selected nucleotide repeat sequence on the candidate pathogen genome or lying within a distance of predefined number of bases with respect to the selected nucleotide repeat sequence on the genome of the pathogen wherein, the important functional genes refer to the genes in pathogens which encode for proteins which are critical for survival, pathogenicity, interaction with the host, adherence to the host or for the virulence of bacteria, wherein the minimum predefined number of genes to be considered in genomic neighborhood is 10.

14. The method according to claim 1, wherein the non-culturable taxonomic groups or pathogens within a sample collected from an environment is obtained by amplification of marker genes like 16S rRNA within bacteria.

15. The method according to claim 1, wherein the information and detection of non-culturable taxonomic groups or pathogens within a sample is obtained by the binning of whole genome sequencing reads into various taxonomic groups using different methods including sequence similarities as well as several methods using supervised and unsupervised classifiers for taxonomic binning of metagenomics sequences.

16. The method according to claim 1, wherein the distant locations refer to distance of greater than 10000 nucleotide base pairs.

17. The method according to claim 1, wherein the sequence matching is performed by processor implemented tools for nucleotide sequence alignment which comprise PILER, BLAST or Burrows wheeler alignment tool.

18. The method according to claim 1, wherein the pathogens is identified by amplification of marker genes like 16S rRNA and obtaining their abundance.

19. The method according to claim 1, wherein the taxonomic constitution of the sample is obtained from these 16S rRNA sequences using standardized methodologies, wherein the taxonomic constitution is utilized to determine occurrence of pathogens in the samples.

20. A system for combating infections due to Mycobacterium tuberculosis, the system comprises:
a sample collection module for obtaining a sample from an infected area;
a pathogen detection and DNA extraction module isolating DNA from the obtained sample using one of a laboratory methods;
a sequencer for sequencing the isolated DNA;
one or more hardware processors;
a memory in communication with the one or more hardware processors, wherein the one or more first hardware processors are configured to execute programmed instructions stored in the one or more first memories, to:
identify a set of nucleotide repeat sequences in the sequenced DNA which are occurring more than a predefined number of times in the Mycobacterium Tuberculosis;
identify a set of neighborhood genes present upstream and downstream of the set of nucleotide repeat sequences;
annotate the set of neighborhood genes according to their functional roles in their respective pathogen based on their involvement in pathways in the identified set of neighborhood genes;
test the presence of a secondary structure in the identified set of nucleotide repeat sequences;
an administration module configured to prepare and administer an engineered polynucleotide construct on the infected area to combat the infections due to the Mycobacterium tuberculosis, wherein the engineered polynucleotide construct is comprising:
one or more of a set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001,
a first enzyme capable of nicking and cleaving the identified set of nucleotide repeat sequences, and
a second enzyme capable of removal of a set of neighborhood genes flanking the set of nucleotide repeat sequences; and
an efficacy module configured to
check the efficacy of the administered engineered polynucleotide construct to combat the Mycobacterium tuberculosis after a predefined time period; and
re-administer the engineered polynucleotide construct if the Mycobacterium tuberculosis are still present in the infected area post administering.

21. One or more non-transitory machine readable information storage mediums comprising one or more instructions which when executed by one or more hardware processors cause:
obtaining a sample from an infected area;
isolating and extracting DNA from the obtained sample using one of a laboratory method;
sequencing the isolated DNA using a sequencer;
identifying a set of nucleotide repeat sequences in the sequenced DNA which are occurring more than a predefined number of times in the Mycobacterium Tuberculosis;
identifying a set of neighborhood genes present upstream and downstream of the set of nucleotide repeat sequences;
annotating the set of neighborhood genes according to their functional roles in their respective pathogen based on their involvement in pathways in the identified set of neighborhood genes;
testing the presence of a secondary structure in the identified set of nucleotide repeat sequences;
preparing and administering an engineered polynucleotide construct on the infected area to combat the infections due to the Mycobacterium tuberculosis, wherein the engineered polynucleotide construct is comprising:
one or more of a set of nucleotide repeat sequences with multiple copies dispersed in nucleotide sequences of genomes of Mycobacterium tuberculosis, wherein the set of nucleotide repeat sequences comprises one or more of a Sequence ID 001, and reverse complement of the Sequence ID 001,
a first enzyme capable of nicking and cleaving the identified set of nucleotide repeat sequences, and
a second enzyme capable of removal of a set of neighborhood genes flanking the set of nucleotide repeat sequences;
checking the efficacy of the administered engineered polynucleotide construct to combat the Mycobacterium tuberculosis after a predefined time period; and
re-administering the engineered polynucleotide construct if the Mycobacterium tuberculosis are still present in the infected area post administering.
